(19) United States
(12) Patent Application Publication
(10) Pub. No.: US 2026/0355150 A1
(43) Pub. Date: Nov. 12, 2026

(54) ULOTARONT FOR THE ADJUVANT TREATMENT OF MAJOR DEPRESSIVE DISORDER

(71) Applicant: Sumitomo Pharma America, Inc., Marlborough, MA (US)

(72) Inventors: Nina DEDIC, Marlborough, MA (US); Robert HAYES, Marlborough, MA (US); Justine KENT, Marlborough, MA (US)

(73) Assignee: Sumitomo Pharma America, Inc., Marlborough, MA (US)

(21) Appl. No.: 19/107,093

(22) PCT Filed: Aug. 29, 2023
(86) PCT No.: PCT/US2023/073033

§ 371 Date: Feb. 27, 2025

Related U.S. Application Data
(60) Provisional application No. 63/373,909, filed on Aug. 30, 2022.

(51) Int. Cl. A61K 31/381; A61K 45/06; A61P 25/24

(52) U.S. Cl. CPC A61K 31/381; A61K 45/06; A61P 25/24

(57) ABSTRACT

Provided are antidepressant therapies, methods for augmenting antidepressant therapies, particularly antidepressant therapies that have failed to produce an adequate response, and to patient populations who benefit from such augmentation.

### CROSS REFERENCE TO RELATED APPLICATIONS

This application claims priority to U.S. Provisional Application No. U.S. Ser. No. 63/373,909, filed Aug. 30, 2022, the entire contents of which are hereby incorporated by reference as if fully set forth herein.

### FIELD

The present disclosure relates to antidepressant therapies, to methods for augmenting antidepressant therapies, particularly antidepressant therapies that have failed to produce an adequate response, and to patient populations who benefit from such augmentation.

### BACKGROUND

Major depressive disorder, or MDD, is a common and disabling illness that affects up to 15% of people over the course of their lives. Connolly 2011. The introduction of newer-generation antidepressants has improved the prognosis for these patients greatly, but still only about 50-60% of patients will respond to first-line treatment and only 35-40% will experience a remission of symptoms during an initial 8-week trial. Connolly 2011. Agents recommended as first-line antidepressants include, inter alia, selective serotonin reuptake inhibitors (SSRIs), serotonin and norepinephrine reuptake inhibitors (SNRIs), bupropion and mirtazapine.

Although many guidelines exist to aid in the management of major depressive disorder, recommendations for the treatment of depression that has not responded adequately to anti-depressant medication, including treatment-resistant depression, are more limited. Professional guidelines include the American Psychiatric Association Practice Guideline for Treatment of Patients with Major Depressive Disorder, Clinical Practice Recommendations for Depression published in Malhi 2009, the Canadian Network for Mood and Anxiety Treatments (CANMAT), and Clinical Guidelines for the Management of Major Depressive Disorder in Adults. These guidelines include several recommendations after the failure of an initial antidepressant trial, including the addition of psychotherapy, lithium augmentation, augmentation with second-generation antipsychotics (SGAs), augmentation with triiodothyronine, and switching to another antidepressant. While lithium and thyroid hormone have established efficacy augmenting failed therapy, this is only true for use in combination with tricyclic antidepressants (TCAs), and the trials supporting this use were done in less treatment-resistant patients than those who typically receive TCAs today. Connolly 2011. Of all strategies to augment response to new-generation antidepressants, Connolly 2011 reports that quetiapine and aripiprazole are best supported by the evidence, although neither the cost effectiveness nor the longer-term benefit of these strategies has been established. See also Marcus 2008. The development of adjunctive therapies is complicated by the fact, as reported in the literature, that combination drug effects observed in preclinical efficacy assays in psychiatry have generally not translated to clinical trial results. DeMartinis 2019.

Ulotaront (SEP-363856) is a TAAR1 agonist with 5-HT1A agonist activity currently in Phase 3 clinical trials for the treatment of schizophrenia, having the following chemical structure:

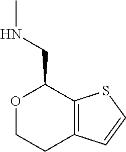

It has a novel mechanism of action (MOA) compared to antipsychotic drugs, as it is not an antagonist at either the dopamine D2 or the serotonin 5-HT2A receptors. Dedic 2019. Ulotaront has demonstrated efficacy in the treatment of symptoms of an exacerbation of schizophrenia in a large randomized, double-blind, placebo-controlled clinical trial, with continued improvement in a 6-month open-label extension study, as reported in US 2020/0179336 A1 (Jun. 11, 2020). Nonclinical studies indicate that agonism at TAAR1 and 5-HT1A receptors contributes to ulotaront's mechanism of action. Dedic 2019.

Ulotaront was identified during a medicinal chemistry program designed to develop structurally and mechanistically novel antipsychotics using in vivo mouse phenotypic screening in combination with comprehensive in vitro and in vivo molecular profiling. As reported by Dedic 2019, this testing revealed that ulotaront was behaviorally active. At 0.3 mg/kg, ulotaront was classified as an anxiolytic but showed a dose-dependent increase in an antipsychotic classification such that the signatures at 1 and 10 mg/kg were predominantly antipsychotic-like. Ulotaront also reportedly showed a modest antidepressant-like signal.

What is needed are therapies for augmenting traditional antidepressant therapy that has yielded an inadequate response in major depressive disorders, and in treatment resistant depression, and criteria for identifying patients who respond to such therapy.

### SUMMARY

As reported in the examples hereto, it has surprisingly been discovered that ulotaront is behaviorally active against depression in a rat model where many traditional antidepressants fail. Thus, in one embodiment, the disclosure provides a method of adjunctively treating MDD (major depressive disorder) in a human subject in need thereof who has exhibited an inadequate response to antidepressant therapy, comprising administering the antidepressant therapy and a therapeutically effective amount of ulotaront or a pharmaceutically acceptable salt thereof to the subject.

In another embodiment, the disclosure provides a method of adjunctively treating a subject suffering from treatment resistant depression comprising administering to the subject antidepressant therapy and a therapeutically effective amount of ulotaront or a pharmaceutically acceptable salt thereof.

Additional methods of treating depression are based on patient characteristics. Thus, in another embodiment, the disclosure provides a method of treating a subject suffering from depression comprising administering to the subject a therapeutically effective amount of ulotaront or a pharmaceutically acceptable salt thereof, wherein the subject has experienced an inadequate response to an antidepressant therapy, and inadequate response is defined as a <50% reduction in MADRS total score from the initiation of antidepressant therapy.

In other aspects, the disclosure relates to methods for the combined treatment of depression using ulotaront and antidepressant therapy. Thus, in another embodiment, the disclosure provides a method of treating a subject suffering from depression (for example, MDD) comprising administering to the subject a therapeutically effective amount of ulotaront or a pharmaceutically acceptable salt thereof and antidepressant therapy.

Additional advantages of the disclosure are set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

All published documents cited herein are hereby incorporated herein by reference in their entirety.

### Use of Terms

As used herein, the singular forms “a”, “an” and “the” are intended to include the plural forms as well, unless the context clearly indicates otherwise.

Unless otherwise specified, the word “includes” (or any variation thereon, e.g., “include”, “including”, etc.) is intended to be open-ended. For example, “A includes 1, 2 and 3” means that A includes but is not limited to 1, 2 and 3.

As used in this specification and in the claims which follow, the word “comprise” and variations of the word, such as “comprising” and “comprises,” means “including but not limited to,” and is not intended to exclude, for example, other additives, components, integers or steps. When an element is described as comprising a plurality components, steps or conditions, it will be understood that the element can also be described as comprising any combination of such plurality, or “consisting of” or “consisting essentially of” the plurality or combination of components, steps or conditions.

When ranges are given by specifying the lower end of a range separately from the upper end of the range, or particular numerical values are specified, it will be understood that a range can be defined by selectively combining any of the lower end variables, upper end variables, and particular numerical values that are mathematically possible. In like manner, when a range is defined as spanning from one endpoint to another, the range will be understood also to encompass a span between and excluding the two endpoints, including any and all ranges and subranges therein. The range is understood to encompass each discrete point within the range as if the same were fully set forth herein.

The present disclosure describes various embodiments. A person of ordinary skill in the art reviewing the disclosure will readily recognize that various embodiments can be combined in any variation. For example, embodiments of the disclosure include treatment of various disorders, patient populations, administrations of dosage forms, at various dosages, minimization of various adverse events, and improvements in various efficacy measures, etc. Any combinations of various embodiments are within the scope of the disclosure.

When published test methodologies and diagnostic instruments are referred to herein, it will be understood that the test methodology or diagnostic instrument is performed based on the version in effect on Jul. 1, 2022, unless otherwise stated to the contrary herein. This is true even when the methodology or instrument is defined herein based on a publication reporting an earlier version.

Unless defined otherwise, all technical and scientific terms used herein have the same meaning as commonly understood by one of ordinary skill in the art to which this disclosure belongs.

### DEFINITIONS

“Adjunctive MDD therapy” refers to the addition of an agent to an antidepressant regimen in order to improve efficacy, because the subject has experienced an inadequate response to an antidepressant regimen that has been optimized in dose and duration.

As used herein, “administering” or “administration” of ulotaront, or a pharmaceutically acceptable salt thereof, encompasses the delivery to a subject of ulotaront, or a pharmaceutically acceptable salt thereof, or a prodrug or other pharmaceutically acceptable derivative thereof, using any suitable formulation or route of administration, e.g., as described herein.

The “AIMS” (Abnormal Involuntary Movement Scale) assessment consists of 10 items describing symptoms of dyskinesia. Guy 1976. Facial and oral movements (items 1 through 4), extremity movements (items 5 and 6), and trunk movements (item 7) are observed unobtrusively while the subject is at rest; the investigator also makes global judgments on the subject's dyskinesias (items 8 through 10). Each item is rated on a 5-point scale, with a score of zero representing absence of symptoms (for item 10, no awareness), and a score of 4 indicating a severe condition (for item 10, awareness, severe distress). In addition, the AIMS includes 2 yes/no questions that address the subject's dental status. The AIMS Movement Rating Score is defined as the sum of items 1 through 7 (i.e., items 1 through 4, facial and oral movements; items 5 and 6, extremity movements; and item 7, trunk movements).

An “antidepressant regimen” or “antidepressant therapy” refers to any pharmacological therapeutic regimen administered as therapy for the treatment of depression, and should be distinguished from augmentation, which is the addition of an agent-not thought to be an antidepressant itself-to an antidepressant regimen in order to improve efficacy. Examples of pharmacological agents for the treatment of depression include, without limitation, SSRIs, SNRIs, bupropion and mirtazapine, and their pharmaceutically acceptable salts.

“Anxious distress” is used herein in accordance DSM-5 (Diagnostic and Statistical Manual of Mental Disorders, Fifth Edition). Thus, a subject with anxious distress must have 2 of the following 5 symptoms for the majority of a major depressive episode: 1) feeling keyed up or tense, 2) feeling unusually restless, 3) difficulty concentrating because of worry, 4) fear that something awful might happen, and 5) a feeling that one might lose control of himself/herself. 0 or 1 symptom=no anxious distress, 2 symptoms=mild anxious distress, 3 symptoms=moderate anxious distress, and 4-5 symptoms=moderate to severe anxious distress (psychomotor agitation must be present).

As used herein, an “at risk” individual is an individual who is at risk of developing a disorder to be treated. This may be shown, for example, by one or more risk factors, which are measurable parameters that correlate with development of a disorder and are known in the art. When a method is said to treat a condition without causing or eliciting an adverse event, it will be understood that the methods can be performed in patients at risk of the adverse event.

The “BARS” (Barnes Akathisia Rating Scale) is a global clinical assessment of akathisia. Barnes 1989. The BARS consists of 4 items related to akathisia: objective observation of akathisia by the investigator, subjective feelings of restlessness by the subject, subjective distress due to akathisia, and global clinical assessment of akathisia. The first 3 items are rated on a 4-point scale, with a score of zero representing absence of symptoms and a score of 3 representing a severe condition. The global clinical evaluation is made on a 6-point scale, with zero representing absence of symptoms and a score of 5 representing severe akathisia.

The “CGI-C” (Clinical Global Impression-Change) Scale measures efficacy of drug treatment by rating the subject's total change from the initiation of drug therapy whether or not it is due entirely to drug treatment. Response choices include: 1=very much improved, 2=much improved, 3=minimally improved, 4=no change, 5=minimally worse, 6=much worse, and 7=very much worse.

The “CGI-S” (Clinical Global Impression-Severity) Scale is a standardized, clinician-administered global rating scale that measures disease severity on a 7-point Likert scale. A higher score on the CGI-S represents a higher severity of disease. To perform this assessment, the rater or investigator answers the following question: “Considering your total clinical experience with this particular population, how mentally ill is the patient at this time?” Response choices include: 1=normal, not at all ill; 2=borderline mentally ill; 3=mildly ill; 4=moderately ill; 5=markedly ill; 6=severely ill; and 7=among the most extremely ill patients.

As used herein, the term “clinically significant” or “clinically meaningful” means an improvement in symptoms which is both statistically significant, and meaningful from a patient's, clinician's, or caregiver's perspective, typically based on a static measure such as CGI-S or a retrospective evaluation of improvement such as the CGI-C, as described generally in various publications of the United States Food and Drug Administration, including FDA 2018, FDA 2019, and FDA 2020. When a treatment or benefit is described herein, it will be understood that in one embodiment, the treatment or benefit shows clinically significant efficacy in a population of patients to a degree of statistical significance.

The “CSFQ-14” is a 14-item short form questionnaire version of the CSFQ (Changes in Sexual Functioning Questionnaire) that produces an overall measure of sexual functioning as well as scores on 2 sets of subscales: a set of 5 scales corresponding to important dimensions of sexual functioning and a set of three scales corresponding to the three phases of the sexual response cycle. Keller 2006.

As used herein, “delaying” development of a disorder means to defer, hinder, slow, stabilize, and/or postpone development of the disorder. Delay can be of varying lengths of time, depending on the history of the disease and/or the individual being treated.

“Depression” has the meaning normally ascribed to the term in the field of psychiatric medicine and can assume the definition set forth in DSM-5. When depression is referred to herein, it will be understood that MDD is a species of depression, and that the disclosure in all aspects is more specifically targeted at the treatment of MDD.

“DSM-5” refers to the Diagnostic and Statistical Manual of Mental Disorders, Fifth Edition. Terms used herein can be defined by reference to DSM-5 when necessary to give life and meaning to the term. When a person is defined in this document based on DSM-5, it will be understood that the person need not have been diagnosed using the criteria in DSM-5, but that the person would meet the criteria specified in DSM-5 if so diagnosed.

The “FAST” (Functional Assessment Short Test) is a 24-item questionnaire designed to assess to what extent the subject is experiencing difficulties in various aspects of functioning. The subjects rate their level of difficulty as 0 (no difficulty), 1 (mild difficulty), 2 (moderate difficulty), and 3 (severe difficulty).

The “HAM-A” (Hamilton Anxiety Rating Scale) evaluates the anxiety symptoms of a patient and in one embodiment, it is administered using the Structured Interview Guide for the Hamilton Anxiety Rating Scale (SIGH-A). Williams 2008.

The “HAM-D17” (17-item Hamilton Depression Rating Scale) is another well-known instrument for evaluating a subject's level of depression and is administered utilizing the Structured Interview Guide for the Hamilton Depression Rating Scale (SIGH-D). Williams 1988.

“Inadequate response,” unless otherwise specified, refers to the lack of a clinically significant improvement in the subject's depression symptoms from the initiation of therapy. Inadequate response is always based on an adequate or therapeutically effective dose of antidepressant therapy for a therapeutically accepted period of time. In some embodiments, inadequate response is defined as a <50%, <40%, <30%, or <20% reduction in MADRS total score from the initiation of therapy, and/or a CGI-C score of ≥3 from the initiation of therapy. In one embodiment, inadequate response is defined as a <50% reduction in MADRS total score from the initiation of therapy. In another embodiment, inadequate response is defined as a CGI-C score of ≥3. In yet another embodiment, inadequate response is defined as a <50% reduction in MADRS total score from the initiation of therapy and a CGI-C score of ≥3. In one embodiment, inadequate response is defined as a <50% improvement in symptom severity per MGH-ATRQ (Massachusetts General Hospital Antidepressant Treatment Response Questionnaire). In another embodiment, inadequate response is defined as a <50% reduction in the HAM-D17 (17-item Hamilton Depression Rating Scale) total score. In another embodiment, inadequate response is defined as a HAM-D17 total score≥14.

The “MADRS” (Montgomery-Asberg Depression Rating Scale) is a well-known instrument for evaluating a subject's level of depression. Montgomery 1979. The MADRS consists of 10 items, each rated from 0 to 6. A higher score on the MADRS represents a higher severity of the level of depression.

A “Major Depressive Episode” or “MDE” has the definition ascribed to the term in DSM-5. Thus, (i) the patient must have 5 or more depressive symptoms for ≥2 weeks; (ii) the patient must have either depressed mood or loss of interest/pleasure; (iii) the symptoms must cause significant distress or impairment; and (iv) the patient has never had a manic or hypomanic episode. Depressive symptoms are selected from the group consisting of: (i) depressed mood, (ii) markedly diminished interest or pleasure in most or all activities, (iii) significant weight loss (or poor appetite) or weight gain, (iv) insomnia or hypersomnia, (v) psychomotor retardation, (vi) fatigue or loss of energy, (vii) feelings of worthlessness or excessive or inappropriate guilt, (viii) diminished ability to think or concentrate, or indecisiveness, and (ix) recurrent thoughts of death (not just fear of dying), or suicidal ideation, plan, or attempt.

The “PGI-C” (Patient Global Impression—Change) Scale measures efficacy of drug treatment by rating the subject's total change from the initiation of drug therapy whether or not it is due entirely to drug treatment, from the patient's perspective.

The “PGI-S” (Patient Global Impression—Severity) is a single-item report of the subject's severity of symptoms. Subjects are asked: “Please choose the response below that best describes the severity of your depression over the past week.” Representative answers include non-overlapping responses such as none, mild, moderate, and severe.

“Pharmaceutically acceptable” or “physiologically acceptable” refer to compounds, salts, compositions, dosage forms and other materials which are useful in preparing a pharmaceutical composition that is suitable for veterinary or human pharmaceutical use.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et. al., describe pharmaceutically acceptable salts in detail in J. Pharmaceutical Sciences, 1977, 66, 1-19. Pharmaceutically acceptable salts of ulotaront include those derived from suitable inorganic and organic acids and bases.

Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemi sulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like. Although pharmaceutically acceptable counter ions are typically used for preparing pharmaceutical formulations, other anions (X) are quite acceptable as synthetic intermediates. Thus, X may be pharmaceutically undesirable anions, such as iodide, oxalate, trifluoromethanesulfonate and the like, when such salts are chemical intermediates.

As used herein, the term “pharmaceutically acceptable excipient” includes, without limitation, any binder, filler, adjuvant, carrier, excipient, glidant, sweetening agent, diluent, preservative, dye/colorant, flavor enhancer, surfactant, wetting agent, dispersing agent, suspending agent, stabilizer, isotonic agent, solvent, emulsifier, anti-caking agent, flavor, desiccants, plasticizers, disintegrants, lubricant, polymer matrix system, and polishing agents, which has been approved by or is otherwise acceptable to the United States Food and Drug Administration upon proper qualification for use in humans or domestic animals.

As used herein, “prevention” or “preventing” refers to a regimen that protects against the onset of the disorder such that the clinical symptoms of the disorder do not develop. Accordingly, “prevention” relates to administration of a therapy to a subject before signs of the diseases are detectable in the subject (for example, administration of a therapy in the absence of a detectable symptom of the disorder). The subject may be an individual at risk of developing the disorder.

The “PSQI” (Pittsburgh Sleep Quality Index) contains 19 self-rated questions and 5 questions rated by the bed partner or roommate (if one is available). Only self-rated questions are included in the scoring. The 19 self-rated items are combined to form 7 “component” scores, each of which has a range of 0 to 3 points. In all cases, a score of “0” indicates no difficulty, while a score of “3” indicates severe difficulty. The 7 component scores are then added to yield one “global” score, with a range of 0 to 21 points, “0” indicating no difficulty and “21” indicating severe difficulties in all areas.

“Remission” of MDD, unless otherwise specified, means that the subject no longer suffers from MDD, e.g., as defined by DSM-5. Alternatively, in other embodiments, remission can be defined based on a MADRS total score≤10, and ≥50% reduction in MADRS total score from the initiation of therapy.

“Response” to therapy, unless otherwise specified, refers to a clinically significant improvement in the subject's diagnosis from the initiation of therapy. Alternatively, response can be defined as ≥50% reduction in MADRS total score from the initiation of therapy. Alternatively, response can be defined as a CGI-C score of 1 or 2 (very much improved or much improved) from the initiation of therapy. Alternatively, response can be defined as ≥50% reduction in MADRS total score from the initiation of therapy and a CGI-C score of 1 or 2 (very much improved or much improved) from the initiation of therapy.

The “SAS” (Simpson Angus Scale) consists of a list of 10 symptoms of Parkinsonism (gait, arm dropping, shoulder shaking, elbow rigidity, wrist rigidity, head rotation, glabella tap, tremor, salivation, and akathisia). Simpson 1970. Each item is rated on a 5-point scale, with a score of zero representing absence of symptoms, and a score of 4 representing a severe condition. The SAS Total Score is the sum of the scores for all 10 items.

The “SF-36” (36-Item Short-form Survey) is a self-reported questionnaire with a standard recall period of 4 weeks which measures generic health-related quality of life on 2 broad domains, physical and mental composites, across 8 health domain scales: physical functioning, pain, role physical, general health, vitality/fatigue, social functioning, role emotional, and mental health. The SF-36 uses norm-based scoring to generate scores on a scale of 0 to 100 where lower scores on the physical component summary and mental component summary represent a lower health-related quality of life and a score of 50 references the normative data derived from surveys of representative samples of US general population. In addition to the composite scores and the individual health domain scores, the SF-36 provides a risk for depression score and the SF-6D health utility index on a scale from 0.0 (worst measured health state) to 1.0 (best measured health state).

As used herein, the term “significantly” refers to a level of statistical significance. The level of statistical significance can be p<0.1, p<0.05, p<0.01, p<0.005, or p<0.001. Unless otherwise specified, the level of statistical significance when the term “significant,” “significantly,” or other variations of the term are used is p<0.05. When a measurable result or effect is expressed or identified herein, it will be understood that the result or effect is typically evaluated based upon its statistical significance relative to a baseline such as placebo. In like manner, when a treatment or benefit is described herein, it will be understood that the treatment or benefit typically shows efficacy in a population of patients to a degree of statistical significance.

“SNRIs” (Serotonin-norepinephrine Reuptake Inhibitors) include, without limitation, desvenlafaxine (Pristiq®), duloxetine (Cymbalta®), levomilnacipran (Fetzima®), and venlafaxine (Effexor® XR), and their pharmaceutically acceptable salts.

“SSRIs” (Selective Serotonin Reuptake Inhibitors) include, without limitation, citalopram (Celexa®), escitalopram (Lexapro®), fluoxetine (Prozac®), paroxetine (Paxil®, Pexeva®), and sertraline (Zoloft®), and their pharmaceutically acceptable salts.

As used herein, “subject” or “patient” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys.

As used herein, the term “therapeutically effective amount” or “effective amount” refers to an amount that is effective to elicit the desired biological or medical response, including the amount of a compound that, when administered to a subject for treating a disorder, is sufficient to accomplish such treatment of the disorder. The effective amount will vary depending on the disorder, and its severity, and the age, weight, etc. of the subject to be treated. The effective amount may be in one or more doses (for example, a single dose or multiple doses may be required to achieve the desired treatment endpoint). An effective amount may be considered to be given in an effective amount if, in conjunction with one or more other agents, a desirable or beneficial result may be or is achieved. Suitable doses of any co-administered compounds may optionally be lowered due to the combined action, additive or synergistic, of the compound.

As used herein, the terms “treatment,” “treat,” and “treating” refer to reversing, alleviating, delaying the onset of, or inhibiting the progress of a disease or disorder, or one or more symptoms thereof, including but not limited to therapeutic benefit. In some embodiments, treatment is administered after one or more symptoms have developed, for example, acute exacerbation of symptoms. In some embodiments, treatment may be administered in the absence of symptoms. For example, treatment may be administered to a subject prior to the onset of symptoms (e.g., in light of a history of symptoms and/or in light of genetic or other susceptibility factors). Treatment may also be continued after symptoms have resolved, for example to prevent or delay their recurrence.

When treatment with two separate agents is described herein, it will be understood that the agents will be administered concomitantly, based on concurrently running dosing regimens, which may differ in dosing frequency or time of administration. The two agents do not necessarily have to be administered at the same time for the administration to be concomitant. For example, concomitant administration may include one agent that is administered three times per day and one agent that is administered once a day.

Therapeutic benefit includes eradication and/or amelioration of the underlying disorder being treated; it also includes the eradication and/or amelioration of one or more of the symptoms associated with the underlying disorder such that an improvement is observed in the subject, notwithstanding that the subject may still be afflicted with the underlying disorder.

In some embodiments, “treatment” or “treating” includes one or more of the following: (a) inhibiting the disorder (for example, decreasing one or more symptoms resulting from the disorder, and/or diminishing the extent of the disorder); (b) slowing or arresting the development of one or more symptoms associated with the disorder (for example, stabilizing the disorder and/or delaying the worsening or progression of the disorder); and/or (c) relieving the disorder (for example, causing the regression of clinical symptoms, ameliorating the disorder, delaying the progression of the disorder, and/or increasing quality of life.)

“Treatment resistant depression” or “treatment failure” have the same clinical criteria and will assume the U.S. FDA regulatory definition of the term unless otherwise specified herein, i.e., a lack of clinically meaningful improvement (≤25% in MADRS total score) in the current episode of depression after treatment with at least 2 different antidepressant agents prescribed in adequate dosages for an adequate duration (at least 6 weeks). Alternatively, the term can be defined as having ≤25% improvement in MADRS total score to 2 or 3 prior antidepressants despite adequate dosage and duration as documented on the Massachusetts General Hospital-Antidepressant Treatment Response Questionnaire (ATRQ).

“Ulotaront,” as referred to herein for use in the methods of the present disclosure, has the chemical name(S)-(4,5-dihydro-7H-thieno[2,3-c]pyran-7-yl)-N-methylmethanamine (which may be abbreviated as “(S)-TPMA”). Ulotaront has the following structure:

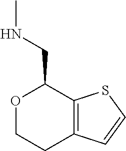

Unless stated otherwise, or unless context requires otherwise, for purposes of this disclosure, the term “ulotaront,” standing alone, includes the free form of ulotaront and also includes its pharmaceutically acceptable salts, hydrates, solvates, amorphous and crystalline forms. When the free form is intended, or any other form or salt is specifically intended, it will be stated as such expressly.

Ulotaront can be used in the methods described herein as the free base (free form) or in the form of a pharmaceutically acceptable salt. In some embodiments, a hydrochloric acid (HCl) salt of ulotaront is used in the methods described herein. Ulotaront, or a pharmaceutically acceptable salt thereof, including its HCl crystalline forms, can be obtained according to the production methods described in PCT Patent Publication No. WO2011/069063 (U.S. Pat. No. 8,710,245, issued Apr. 29, 2014) or PCT Patent Publication No. WO2019/161238, which are incorporated herein by reference in entirety and for all purposes, or a method analogous thereto.

Also provided herein are pharmaceutical compositions and dosage forms, comprising ulotaront, or a pharmaceutically acceptable salt thereof, and one or more pharmaceutically acceptable excipients. Compositions and dosage forms provided herein may further comprise one or more additional active ingredients. Ulotaront, or a pharmaceutically acceptable salt thereof, may be administered as part of a pharmaceutical composition as described herein.

### Discussion

In one embodiment, the disclosure provides a method of adjunctively treating MDD (major depressive disorder) in a human subject in need thereof who has exhibited an inadequate response to antidepressant therapy, for example, after at least 8 weeks of antidepressant therapy, comprising administering the antidepressant therapy and a therapeutically effective amount of ulotaront or a pharmaceutically acceptable salt thereof to the subject.

In another embodiment, the disclosure provides a method of adjunctively treating a subject suffering from treatment resistant depression comprising administering to the subject antidepressant therapy and a therapeutically effective amount of ulotaront or a pharmaceutically acceptable salt thereof.

In another embodiment, the disclosure provides a method of treating a subject suffering from depression comprising administering to the subject a therapeutically effective amount of ulotaront or a pharmaceutically acceptable salt thereof wherein the subject has experienced an inadequate response to an antidepressant therapy, and inadequate response is defined as a <50% reduction in MADRS total score from the initiation of therapy. In one embodiment, the method is adjunctive to an antidepressant regimen comprising antidepressant therapy, in a subject who has exhibited an inadequate response to the antidepressant therapy, e.g., after at least eight weeks of the antidepressant therapy. In another embodiment, inadequate response is further defined as a CGI-C score of ≥3 from the initiation of therapy.

In another embodiment, the disclosure provides a method of treating a subject suffering from depression (for example, MDD) comprising administering to the subject a therapeutically effective amount of ulotaront or a pharmaceutically acceptable salt thereof and antidepressant therapy.

In any of the embodiments of the current disclosure, the antidepressant therapy comprises an SSRI (Selective Serotonin Reuptake Inhibitor) or an SNRI (Serotonin-norepinephrine Reuptake Inhibitor). In some embodiments, antidepressant therapies comprise an SSRI selected from escitalopram 10 to 20 mg/day, fluoxetine 20 to 60 mg/day, paroxetine 25 to 62.5 mg/day, or sertraline 50 to 200 mg/day; or a SNRI selected from duloxetine 30 to 60 mg/day and venlafaxine 37.5 to 225 mg/day. The foregoing therapies are generally considered to be “adequate therapies” when tailored to a particular subject. In any of the embodiments of the current disclosure, the subject has experienced an inadequate response or treatment failure to the antidepressant therapies mentioned in this paragraph.

Any of the embodiments of the current disclosure can further be defined by the subject to be treated. Thus, in one embodiment the subject is suffering from a major depressive episode (MDE), e.g., as defined by DSM-5. The MDE can be of varying lengths, including ≥4 weeks, ≥8 weeks, or ≥26 weeks in duration. In some embodiments, the MDE is ≥8 weeks in duration and ≤52 weeks in duration. In another embodiment, the subject is in a current major depressive episode (MDE), as defined by DSM-5 (Diagnostic and Statistical Manual of Mental Disorders, Fifth Edition) criteria and confirmable by the Massachusetts General Hospital Antidepressant Treatment Response Questionnaire (MGH-ATRQ).

In another embodiment, the subject is characterized by an inadequate response to at least 1 and no more than 3 adequate antidepressant therapies (ADTs) during the current MDE. In some embodiments the antidepressant therapy comprises an SSRI or SNRI, or one of the therapies aforementioned in this document.

In still other embodiments the subject is characterized by his or her degree of depression prior to the ADT. Thus, in another embodiment, the subject has a 17-item Hamilton Depression Rating Scale (HAM-D17) total score≥18, but optionally ≥14, ≥16, ≥20, or ≥22 prior to the ADT.

In still further embodiments the subject treated, prior to ADT, will satisfy all three of the foregoing requirements for MDE status of at least 8 weeks, inadequate response to an earlier course of antidepressant therapy, and a HAM-D17 total score of ≥18.

In other embodiments, the subject has an inadequate response after at least 8 weeks of antidepressant therapy, defined as: (a) a <50% reduction in the HAM-D17 (17-item Hamilton Depression Rating Scale) total score; or (b) a HAM-D17 total score≥14; or (c) a CGI-C (Clinical Global Impression—Change) Scale score of ≥3; or (d) a <50% reduction in MADRS (Montgomery Asberg Depression Rating Scale) total score; or (e) a combination of any two or more of (a)-(d).

Other embodiments are defined based on the patient's treatment response measured by MADRS. Thus, in one embodiment, inadequate response is defined as ≤25% reduction in MADRS total score. In another embodiment, inadequate response is defined as <50% and ≥25% reduction in MADRS total score.

Still further embodiments find particular utility in subjects with anxious distress and are based on the subject's level of anxiety. Thus, any of the methods of the current disclosure can be practiced in subjects having anxious distress, as defined in DSM 5.

The methods of the current disclosure can also be defined based on the dose of ulotaront administered. Thus, in one embodiment, the therapeutically effective amount comprises from 10 to 150 mg, from 15 to 125 mg, from 25 to 100 mg, from 25 to 75 mg, or from 50 to 100 mg of ulotaront or a pharmaceutically acceptable salt thereof, based on the weight of the free form of ulotaront. In another embodiment, the therapeutically effective amount comprises 50 or 75 mg of ulotaront or a pharmaceutically acceptable salt thereof, based on the weight of the free form of ulotaront. In still further embodiments, the therapeutically effective amount comprises 20, 25, 30, 35, 40, 45, 50, 55, 60, 65, 70, 75, 80, 85, 90, 95, or 100 mg of ulotaront or a pharmaceutically acceptable salt thereof, based on the weight of the free form of ulotaront.

In any of these embodiments, the therapeutically effective amount of ulotaront is administered once daily for a period of at least six weeks.

The methods of the disclosure can further be defined based on its effect on the disease. Thus, any of the methods of the current disclosure can produce remission of the depression, including remission of the depression wherein the depression is defined according to DSM 5 (Diagnostic and Statistical Manual of Mental Disorders, Fifth Edition).

Any of the methods of the disclosure can further produce a mean change in the subject's Montgomery-Asberg Depression Rating Scale. In various embodiments, the methods can reduce the MADRS total score greater than antidepressant therapy alone by ≥1, ≥2, ≥3, ≥4, or ≥5 points. In other embodiments, the treatment improves the subject's MADRS total score. In other embodiments, the treatment improves the subject's CGI-S score of ≥2, ≥3, or ≥4 points. In still further embodiments the treatment produces a clinically significant improvement in the subject's MADRS total score and CGI-S score.

Other embodiments can be defined based on alternative endpoints. Thus, in various embodiments the method improves the subject's FAST (Functional Assessment Short Test) score.

In still further embodiments the method improves the subject's PGI-S(Patient Global Impression—Severity) Score.

In other embodiments, the method improves the subject's SF-36 (36-Item Short form Survey) score.

In further embodiments, the method improves the subject's HAM-A (Hamilton Anxiety Rating Scale) total score.

In still further embodiments, the method improves the subject's HAM D17 (17-item Hamilton Depression Rating Scale) total score.

In other embodiments, the method produces a CGI-C (Clinical Global Impression—Change) score of 1 or 2 (very much improved or much improved).

In further embodiments, the method reduces the subject's MADRS total score by ≥50%. In still further embodiments, the method reduces the subject's MADRS total score to ≤10.

In another embodiment, the method reduces the subject's MADRS total score to ≤10 and reduces the subject's MADRS total score by ≥50%.

The method also can be defined based on its period of administration, typically improving the subject's depression symptoms at least until the end of the period. Thus, any of the embodiments of the disclosure can be practiced by administering the ulotaront or pharmaceutically acceptable salt and improving the subject's depression symptoms for a therapeutically effective period of time of 6 weeks or more, 8 weeks or more, 12 weeks or more, 18 weeks or more, 26 weeks or more, or 52 weeks or more. The improvement is represented, e.g., by a reduction in the subject's MADRS score (i.e., a ≥25% reduction, ≥40% reduction, or ≥50% reduction), a resulting MADRS total score of ≤14, ≤12, ≤10, or ≤8, or a combination of score reduction and absolute total score, for example, a ≤50% reduction and a total score≤10.

In another embodiment, the improvement is represented by a ≥50% reduction in the subject's MADRS score a resulting MADRS total score of ≤10, and is observed for a therapeutically effective period of time of at least 26 weeks.

The methods of the disclosure can also be defined based on their excellent side effect profile. Thus, in various embodiments, the method results in no clinically significant worsening of the subject's: (a) abnormal involuntary movements as measured by the AIMS (Abnormal Involuntary Movement Scale); or (b) akathisia, e.g., as measured by the BARS (Barnes Akathisia Rating Scale); or (c) sexual functioning, e.g., as measured by the CSFQ-14 (Changes in Sexual Functioning Questionnaire, Short Form); or (d) suicidality, e.g., as measured by the C SSRS (Columbia-Suicide Severity Rating Scale); or (e) Parkinsonism, e.g., as measured by the SAS (Simpson Angus Scale); or (f) sleep quality, e.g., as measured by the PSQI (Pittsburgh Sleep Quality Index); or (g) any combination of two or more of (a)-(f).

Despite their excellent side effect profile, the methods are optionally further monitored for the emergence of any potential side-effects. In some embodiments, the methods further comprise monitoring for CNS (central nervous system) stimulation including agitation, restlessness, insomnia, depression including lethargy, somnolence, and shallow breathing, impaired muscle coordination, effects on heart rate, and change in pupil size.

Certain aspects of the disclosure can be defined based on Embodiments AA through BW below:

[Embodiment AA] A method of adjunctively treating MDD (major depressive disorder) in a human subject in need thereof who has exhibited an inadequate response to antidepressant therapy, comprising administering the antidepressant therapy and a therapeutically effective amount of ulotaront or a pharmaceutically acceptable salt thereof to the subject.

[Embodiment AB] A method of adjunctively treating a human subject suffering from treatment resistant depression comprising administering to the subject antidepressant therapy and a therapeutically effective amount of ulotaront or a pharmaceutically acceptable salt thereof.

[Embodiment AC] The method of embodiment AB, wherein treatment resistant depression is defined as treatment failure to more than one antidepressant therapy.

[Embodiment AD] The method of embodiment AA, wherein inadequate response is defined, after at least 8 weeks of antidepressant therapy, as <50% reduction in depressive symptom severity, as assessed by the Massachusetts General Hospital Antidepressant Treatment Response Questionnaire (MGH-ATRQ).

[Embodiment AE] A method of treating a human subject suffering from depression comprising administering to the subject a therapeutically effective amount of ulotaront or a pharmaceutically acceptable salt thereof, wherein the subject has experienced an inadequate response to an antidepressant therapy, and inadequate response is defined as a <50% reduction in MADRS total score from the initiation of antidepressant therapy.

[Embodiment AF] A method of treating a human subject suffering from depression comprising administering to the subject a therapeutically effective amount of ulotaront or a pharmaceutically acceptable salt thereof, wherein the subject has experienced an inadequate response to an antidepressant therapy, and inadequate response is defined as a <50% reduction in MADRS total score from the initiation of antidepressant therapy, and a CGI-C score of ≥3 from the initiation of therapy.

[Embodiment AG] The method of embodiment AE or AF, wherein the method is adjunctive to an antidepressant regimen comprising antidepressant therapy in a human subject in need thereof who has exhibited an inadequate response to the antidepressant therapy.

[Embodiment AH] The method of any of embodiments AA-AG, wherein the antidepressant therapy comprises an SSRI (Selective Serotonin Reuptake Inhibitor) or an SNRI (Serotonin-norepinephrine Reuptake Inhibitor).

[Embodiment AI] The method of any of embodiments AA-AG, wherein the antidepressant therapy comprises an SSRI selected from escitalopram 10 to 20 mg/day, fluoxetine 20 to 60 mg/day; paroxetine 25 to 62.5 mg/day; and sertraline 50 to 200 mg/day; or an SNRI selected from duloxetine 30 to 60 mg/day and venlafaxine 37.5 to 225 mg/day.

[Embodiment AJ] The method of any of embodiments AA, AD, AH, and AI, wherein inadequate response is defined as a <50% reduction in MADRS total score from the initiation of therapy. [Embodiment AK] The method of any of embodiments AA, AD, AH, and AI, wherein inadequate response is defined as a CGI-C score of ≥3 from the initiation of therapy.

[Embodiment AL] The method of any of embodiments AA and AD-AI, wherein the inadequate response is defined as: (a) (i) a <50% reduction in the HAM-D17 (17-item Hamilton Depression Rating Scale) total score or (ii) a HAM-D17 total score≥14; and (b) a CGI-C (Clinical Global Impression—Change) Scale score of ≥3; and (c) a <50% reduction in MADRS (Montgomery Asberg Depression Rating Scale) total score.

[Embodiment AM] The method of any of embodiments AA and AD-AI wherein the inadequate response is defined as: (a) a <50% reduction in the HAM-D17 (17-item Hamilton Depression Rating Scale) total score; and (b) a HAM-D17 total score≥14; and (c) a CGI-C (Clinical Global Impression—Change) Scale score of ≥3; and (d) a <50% reduction in MADRS (Montgomery Asberg Depression Rating Scale) total score.

[Embodiment AN] The method of any of embodiments AA and AD-AM, wherein the subject has a HAM-D17 (17-item Hamilton Depression Rating Scale) total score≥18 prior to antidepressant therapy, and inadequate response is defined, after at least 8 weeks of antidepressant therapy, as: (a) a <50% reduction in the HAM-D17 (17-item Hamilton Depression Rating Scale) total score; or (b) a HAM-D17 total score≥14; or (c) a CGI-C (Clinical Global Impression—Change) Scale score of ≥3; or (d) a <50% reduction in MADRS (Montgomery Asberg Depression Rating Scale) total score; or (e) a combination of (a)-(d).

[Embodiment AO] The method of any of embodiments AA and AD-AM, wherein the subject has a HAM-D17 (17-item Hamilton Depression Rating Scale) total score≥18 prior to antidepressant therapy, and inadequate response is defined, after at least 8 weeks of antidepressant therapy, as: (a) a <50% reduction in the HAM-D17 (17-item Hamilton Depression Rating Scale) total score; and (b) a HAM-D17 total score≥14; and (c) a CGI-C (Clinical Global Impression—Change) Scale score of ≥3; and (d) a <50% reduction in MADRS (Montgomery Asberg Depression Rating Scale) total score.

[Embodiment AP] The method of any of embodiments AA-AO, wherein the subject is in a current major depressive episode (MDE), as defined by DSM-5 (Diagnostic and Statistical Manual of Mental Disorders, Fifth Edition) criteria.

[Embodiment AQ] The method of any of embodiments AA-AO, wherein the subject is in a current major depressive episode (MDE), as defined by DSM-5 (Diagnostic and Statistical Manual of Mental Disorders, Fifth Edition) criteria, of ≥8 weeks in duration.

[Embodiment AR] The method of any of embodiments AA-AQ, comprising administering the ulotaront or pharmaceutically acceptable salt and improving the subject's depression symptoms for a therapeutically effective period of time of 6 weeks or more, 8 weeks or more, 12 weeks or more, 26 weeks or more, or 52 weeks or more.

[Embodiment AS] The method of any of embodiments AA-AR, wherein the therapeutically effective amount comprises from 25 to 100 mg of ulotaront or a pharmaceutically acceptable salt thereof, based on the weight of the free form of ulotaront.

[Embodiment AT] The method of any of embodiments AA-AR, wherein the therapeutically effective amount comprises 50 or 75 mg of ulotaront or a pharmaceutically acceptable salt thereof, based on the weight of the free form of ulotaront.

[Embodiment AU] The method of any of embodiments AA and AD-AT, wherein the subject has exhibited the inadequate response after at least eight weeks of the antidepressant therapy.

[Embodiment AV] The method of any of embodiments AA-AU, comprising administering the ulotaront or pharmaceutically acceptable salt for a period of at least six weeks.

[Embodiment AW] The method of any of embodiments AA-AV, wherein the method increases the subject's Montgomery-Asberg Depression Rating Scale total score compared to antidepressant therapy alone by ≥1, ≥2, ≥3, or ≥4 points.

[Embodiment AX] The method of any of embodiments AA-AW, wherein the method produces remission of the depression.

[Embodiment AY] The method of any of embodiments AA-AW, wherein the method produces remission of the depression, defined according to DSM 5 (Diagnostic and Statistical Manual of Mental Disorders, Fifth Edition).

[Embodiment AZ] The method of any of embodiments AA-AY, wherein the method improves the subject's MADRS (Montgomery Asberg Depression Rating Scale) total score.

[Embodiment BA] The method of any of embodiments AA-AZ, wherein the method improves the subject's CGI-S(Clinical Global Impression—Severity) score by ≥2, ≥3, or ≥4 points. [Embodiment BB] The method of any of embodiments AA-BA, wherein the method produces a clinically significant improvement in the subject's MADRS (Montgomery Asberg Depression Rating Scale) total score and CGI-S(Clinical Global Impression—Severity) score.

[Embodiment BC] The method of any of embodiments AA-BB, wherein the method improves the subject's FAST (Functional Assessment Short Test) score or PGI-S(Patient Global Impression—Severity) score.

[Embodiment BD] The method of any of embodiments AA-BC, wherein the subject has anxious distress as defined in DSM 5 (Diagnostic and Statistical Manual of Mental Disorders, Fifth Edition).

[Embodiment BE] The method of any of embodiments AA-BD, wherein the method improves the subject's SF-36 (36-Item Short form Survey) score.

[Embodiment BF] The method of any of embodiments AA-BE, wherein the method improves the subject's HAM-A (Hamilton Anxiety Rating Scale) total score.

[Embodiment BG] The method of any of embodiments AA-BF, wherein the method improves the subject's HAM D17 (17-item Hamilton Depression Rating Scale) total score.

[Embodiment BH] The method of any of embodiments AA-BG, wherein the method produces a CGI-C (Clinical Global Impression—Change) score of 1 or 2 (very much improved or much improved).

[Embodiment BI] The method of any of embodiments AA-BH, wherein the method reduces the subject's MADRS total score by ≥50%.

[Embodiment BJ] The method of any of embodiments AA-BI, wherein the method reduces the subject's MADRS total score to ≤10.

[Embodiment BK] The method of any of embodiments AA-BJ, wherein the method reduces the subject's MADRS total score to ≤10 and reduces the subject's MADRS total score by ≥50%. [Embodiment BL] The method of any of embodiments AA-BK, wherein the method results in no clinically significant worsening of the subject's: abnormal involuntary movements as measured by the AIMS (Abnormal Involuntary Movement Scale); or akathisia as measured by the BARS (Barnes Akathisia Rating Scale); or sexual functioning as measured by the CSFQ-14 (Changes in Sexual Functioning Questionnaire, Short Form); or suicidality as measured by the C SSRS (Columbia-Suicide Severity Rating Scale); or Parkinsonism as measured by the SAS (Simpson Angus Scale); or sleep quality as measured by the PSQI (Pittsburgh Sleep Quality Index); or any combination thereof.

[Embodiment BM] The method of any of embodiments AA-BK, wherein the method results in no clinically significant sedation, symptoms of metabolic syndrome (e.g., weight gain, diabetes, hyperlipidemia), or movement disorders.

[Embodiment BN] The method of any of embodiments AA-BL, further comprising monitoring for CNS (central nervous system) stimulation including agitation, restlessness, insomnia, depression including lethargy, somnolence, and shallow breathing, impaired muscle coordination, effects on heart rate, and change in pupil size.

[Embodiment BO] A method of treating a subject suffering from depression (e.g., MDD) comprising administering to the subject a therapeutically effective amount of ulotaront or a pharmaceutically acceptable salt thereof and antidepressant therapy.

[Embodiment BP] The method of embodiment BO, wherein the antidepressant therapy comprises a therapeutically effective amount of an SSRI (Selective Serotonin Reuptake Inhibitor) or an SNRI (Serotonin-norepinephrine Reuptake Inhibitor).

[Embodiment BQ] The method of embodiment BO, wherein the antidepressant therapy comprises an SSRI selected from escitalopram (e.g., 10 to 20 mg/day), fluoxetine (e.g., 20 to 60 mg/day), paroxetine (e.g., 25 to 62.5 mg/day), or sertraline e.g., 50 to 200 mg/day); or a SNRI selected from duloxetine (e.g., 30 to 60 mg/day) and venlafaxine (e.g., 37.5 to 225 mg/day).

[Embodiment BR] The method of any of embodiments AA-BQ, wherein the patient has a HAM-D17 (17-item Hamilton Depression Rating Scale) total score≥14, ≥16, or ≥18 prior to ulotaront therapy.

[Embodiment BS] The method of any of embodiments AA-BR, wherein the patient has a MADRS (Montgomery-Asberg Depression Rating Scale) total score≥20, ≥22, ≥24, ≥26, ≥28, ≥30, ≥32, or ≥34 prior to ulotaront therapy.

[Embodiment BT] The method of any of embodiments AA, and AD-BS, wherein inadequate response is defined as treatment failure.

[Embodiment BU] The method of any of embodiments AA, and AD-BS, wherein the patient does not have treatment resistant depression.

[Embodiment BV] The method of any of embodiments AA, and AD-BS, wherein inadequate response is defined as ≤25% reduction in MADRS total score.

[Embodiment BW] The method of any of embodiments AA, and AD-BS, wherein inadequate response is defined as <50% and ≥25% reduction in MADRS total score.

### EXAMPLES

In the following examples, efforts have been made to ensure accuracy with respect to numbers (e.g., amounts, temperature, etc.) but some errors and deviations should be accounted for. The following examples are put forth so as to provide those of ordinary skill in the art with a complete disclosure and description of how the methods claimed herein are made and evaluated, and are intended to be purely exemplary of the disclosure and are not intended to limit the scope of what the inventors regard as their disclosure.

### Example 1. Effects of Ulotaront in Combination with Antidepressants in the Mouse Forced Swim Test

Ulotaront was evaluated in the mouse forced swim test (FST). In this test, mice are placed in a small tank of water from which they cannot escape, and their behavior is monitored. Acute administration of all major classes of marketed antidepressants decreases the duration of immobility in this test.

These particular FST studies were conducted in male Swiss mice (5-6 weeks of age, n=10 per group), including mice co-administered imipramine, fluoxetine, paroxetine, venlafaxine or citalopram. Single administration of ulotaront (0.3, 1, 3, or 10 mg/kg, po) did not significantly alter immobility time or enhance the antidepressant activity of imipramine, fluoxetine, paroxetine, venlafaxine or citalopram following co-administration at single doses.

These results stand in contrast to the FST results in Male BalbC/J mice reported by Dedic 2019. There, Male BalbC/J mice (6 weeks of age, n=8 per group) received a single administration of vehicle (po), ulotaront (0.3, 1, 3 or 10 mg/kg, po), or sertraline (20 mg/kg, i.p.) as a positive control. Ulotaront treatment significantly reduced immobility duration at doses of 1, 3, and 10 mg/kg with the maximal effect achieved at the 1 mg/kg dose level. The results demonstrate that ulotaront shows antidepressant-like effects in the mouse FST, however this effect is strain specific. Varying sensitivity to established antidepressant drugs has also been reported across different mouse strains (Lucki 2001; David 2003).

### Example 2. Effects of Ulotaront in the Rat Forced Swim Test

The antidepressant-like effects of ulotaront were also examined using two different dosing protocols in the rat FST assay. In both procedures, rats were pre-exposed to a 15 min swim session (day 0) prior to the 5 min FST session which was conducted 24 h later (day 1).

In the first study, adult male Wistar rats (206-260 g; n=12 per group) were dosed on 3 occasions with their assigned treatment administered at 24 h (i.e., immediately after the pre-swim), 4 h and 1 h prior to testing. The treatment groups were as follows: vehicle (po), ulotaront (0.3, 1, 3 or 10 mg/kg po) or imipramine (64 mg/kg, po) as a positive control. The time spent immobile in the 5 min test session was recorded by an observer blinded to treatment conditions and results were analyzed by one way ANOVA followed by Dunnett's post hoc comparisons. Ulotaront significantly reduced the duration of immobility at 3 mg/kg (po) compared to vehicle-treated controls.

In the second study, adult, male Sprague-Dawley rats (190-234 g, n=10 per group) were dosed on only one occasion immediately after the pre-swim i.e., 24 h prior to testing. This dosing protocol has been used to show antidepressant-like effects of acute ketamine treatment whereas other drug classes (e.g., SSRIs and tricyclics) are typically ineffective (Ardalan 2017; Cryan 2005; Koike 2014). Rats were treated with either vehicle (po), ulotaront (0.3, 1, 3 or 10 mg/kg, po) or ketamine (10 mg/kg, i.p.) included as a positive control. Behavior was quantified during the 5 min test session using a time sampling technique where the actual behavior observed, i.e., immobility, climbing or swimming, was recorded every 5 seconds for a total of 60 observations. Results were analyzed by one way ANOVA followed by Dunnett's post hoc comparisons.

Ulotaront significantly reduced the frequency of immobility at all doses tested (Table 1). The magnitude of the effect was similar to ketamine-treated rats and was associated with a significant increase in swimming behavior.

TABLE 1

| | FST |
|---|---|
| Group | Immobility frequency (Mean ± SEM) |
| Vehicle | 44.7 ± 2.4 |
| Ulotaront 0.3 mg/kg, po | 29.6 ± 2.6^{a} |
| Ulotaront 1 mg/kg, po | 30.1 ± 3.0^{a} |
| Ulotaront 3 mg/kg, po | 32.6 ± 2.0^{a} |
| Ulotaront 10 mg/kg, po | 27.0 ± 2.4^{a} |
| Ketamine 10 mg/kg, ip | 22.9 ± 3.5^{a} |

^{a}p < 0.05 compared to vehicle (one way ANOVA with Dunnett’s post hoc test)

### Example 3: Effects of Ulotaront in a Rat Learned Helplessness Model

Ulotaront was evaluated in the rat learned helplessness model which assesses loss of behavioral control over aversive situations and is responsive to antidepressant treatment (Willner 2015). In this model, rats are first exposed to inescapable foot shock to induce a helpless state which is then expressed by a subsequent failure to learn to avoid escapable foot shocks (active avoidance). Treatment with established antidepressants, typically using a sub-chronic regimen, reduces the number of escape failures in helpless rats.

Adult male Wistar rats (186-262 g; n=16-17 per group) were randomly assigned to 6 different groups. Helplessness was induced on Day 1 in 5 of the groups by exposing animals to inescapable foot shocks delivered through an electrified stainless steel grid floor every 15 seconds for 1 h. The remaining group did not receive inescapable shock and served as nonhelpless controls. Approximately 6 h after helplessness induction on Day 1, rats were dosed po with vehicle, the tricyclic antidepressant agent, imipramine (32 mg/kg) or ulotaront (1, 3 or 10 mg/kg). Treatment continued on days 2-5 with all subjects receiving test agent or vehicle twice daily (AM and PM). Active avoidance was tested 60 min after the morning dose on Days 3, 4 and 5. All rats were tested in a 2-compartment active avoidance chamber where delivery of foot shock was signaled by a light cue. The test session began with 5 min of free exploration after which 30 stimulus-shock trials were delivered during a 15 min period. Each trial lasted 30 seconds with an initial 24 second rest period, followed by a 6 second light cue presentation with foot shock delivered during the last 3 seconds. Crossing to the opposite compartment during the first 3 seconds of light cue presentation avoided the shock and was considered an active avoidance. Failure to avoid full delivery of the foot shock (i.e., failing to cross during 3 second shock presentation) was considered an escape failure. Crossings to the opposite compartment of the shuttle box during the 24 second rest period were recorded as inter-trial interval (ITI) crossings. Escape failures and ITI crossings were analyzed by two way repeated measures ANOVA with treatment as a main factor and test session (i.e., day) as the repeated measure. Significant ANOVA results were followed by Dunnett's post-hoc analysis to examine the effect of treatment at each test session compared to vehicle-treated, helpless rats.

Ulotaront dose-dependently reduced the number of escape failures in helpless rats with significant effects observed at 10 mg/kg po on test days 4 and 5. A significant reduction in escape failures was also observed in rats treated with imipramine (Table 2).

TABLE 2

| | Day 3 | Day 4 | Day 5 |
|---|---|---|---|
| Group | (Mean ± SEM) | (Mean ± SEM) | (Mean ± SEM) |
| Non-helpless/Vehicle | 5 ± 1 | 4 ± 2 | 4 ± 2 |
| Helpless/Vehicle | 15 ± 2 | 19 ± 3 | 19 ± 3 |
| Helpless/Ulotaront | 15 ± 2 | 19 ± 3 | 18 ± 3 |
| 1 mg/kg, po bid | | | |
| Helpless/Ulotaront | 12 ± 2 | 17 ± 3 | 17 ± 3 |
| 3 mg/kg, po bid | | | |
| Helpless/Ulotaront | 9 ± 2 | 8 ± 3^{a} | 9 ± 3^{a} |
| 10 mg/kg, po bid | | | |
| Helpless/Imipramine | 3 ± 1 | 3 ± 2^{a} | 2 ± 1^{a} |
| 32 mg/kg, po bid | | | |

^{a}p < 0.05 compared to helpless/vehicle

Analysis of ITI crossings on test days 3, 4 and 5 showed that ITI crossings were not significantly different in non-helpless rats compared to helpless controls (Table 3). Treatment with 10 mg/kg ulotaront significantly increased ITI crossings on day 5 compared to helpless, vehicle-treated controls, but had no effect on ITI crossings on days 3 and 4. This suggests that interpretation of the antidepressant-like effect of ulotaront on day 4 is not confounded by effects on locomotor activity.

TABLE 3

| | Day 3 | Day 4 | Day 5 |
|---|---|---|---|
| Group | (Mean ± SEM) | (Mean ± SEM) | (Mean ± SEM) |
| Non-helpless/Vehicle | 13 ± 2 | 11 ± 2 | 12 ± 2 |
| Helpless/Vehicle | 10 ± 2 | 9 ± 3 | 9 ± 3 |
| Helpless/Ulotaront | 9 ± 2 | 8 ± 3 | 6 ± 1 |
| 1 mg/kg, po bid | | | |
| Helpless/Ulotaront | 10 ± 2 | 6 ± 2 | 4 ± 1 |
| 3 mg/kg, po bid | | | |
| Helpless/Ulotaront | 11 ± 2 | 14 ± 3 | 18 ± 4^{a} |
| 10 mg/kg, po bid | | | |
| Helpless/Imipramine | 17 ± 2 | 12 ± 2 | 15 ± 2 |
| 32 mg/kg, po bid | | | |

^{a}p < 0.05 compared to helpless/vehicle

The results show that ulotaront exhibits antidepressant-like activity in the rat learned helpless model.

### Example 4: a Phase 2/3, Multicenter, Randomized, Double-Blind, Placebo-Controlled Trial of the Safety and Efficacy of Flexible Doses of Ulotaront as Adjunctive Therapy in the Treatment of Adults with Major Depressive Disorder

This is a phase 2/3, multicenter, randomized, double-blind, placebo-controlled, flexible-dose trial designed to assess the safety and efficacy of ulotaront (50 to 75 mg/day) as adjunctive therapy to an assigned open-label antidepressant therapy (ADT) in subjects with MDD who have demonstrated an inadequate response to a prospective 8-week trial of the same assigned open-label ADT.

The trial population will include subjects 18 to 65 years of age, inclusive, at the time of informed consent with a primary diagnosis of MDD, and in a current major depressive episode (MDE), as defined by Diagnostic and Statistical Manual of Mental Disorders, Fifth Edition (DSM-5) criteria and confirmed by the MGH-ATRQ. The current MDE must be ≥8 weeks in duration. Additionally, subjects must have a reported history for the current MDE of an inadequate response to at least 1 and no more than 3 adequate ADTs. Subjects must also have a 17-item Hamilton Depression Rating Scale (HAM-D₁₇) total score≥18 at the screening and baseline visits.

A primary objective of the study is to compare the efficacy of ulotaront (50 to 75 mg/day) to placebo as adjunctive therapy to an assigned open-label ADT in subjects with MDD who demonstrate an inadequate response to a prospective 8-week trial of the same assigned open label ADT. Primary efficacy will be evaluated by change from the end of Phase A (Week 8 visit) to the end of Phase B (Week 14 visit) in the MADRS total score. Efficacy will also be evaluated, inter alia, by change from end of Phase A (Week 8 visit) to end of Phase B (Week 14 visit) in the CGI S.

### REFERENCES CITED

American Psychiatric Association practice guideline for treatment of patients with major depressive disorder, third edition [online]. Available from URL: http://www.psy chiatryonline.com/pracGuide/pracGuideTopic_7.aspx [Accessed 2022 Jul. 17].
Ardalan M, Rafati A H, Nyengaard J R, Wegener G. Rapid antidepressant effect of ketamine correlates with astroglial plasticity in the hippocampus. Br J Pharmacol 2017; 174:483-492.
Barnes T R. A rating scale for drug-induced akathisia. Br J Psychiatry. 1989; 154:672 676.
Cryan J F, Valenton R J, Lucki I. Assessing substrates underlying the behavioral effects of antidepressants using the modified rat forced swimming test. Neurosci Biobehav Rev 2005; 29(4-5):547-569.
David D J, Renard C E, Jolliet P, Hascoet M, Bourin M. Antidepressant-like effects in various mice strains in the forced swimming test. Psychopharmacology (Berl) 2003; 166:373-382.
Dedic N, Jones P G, Hopkins S C, Lew R., Shao L, Campbell J E, Spear K L, Large T H, Campbell U C, Hanania T, Leahy E, & Koblan K S (2019). SEP-363856, a novel psychotropic agent with a unique, non-D2 receptor mechanism of action. Journal of Pharmacology and Experimental Therapeutics, 371(1), 1-14.
DeMartinis, Nicholas; Lopez, Rene N.; Pickering, Eve H.; Schmidt, Christopher J.; Gertsik, Lev; Walling, David; Ogden, Adam. A Proof-of-Concept Study Evaluating the Phosphodiesterase 10A Inhibitor PF-02545920 in the Adjunctive Treatment of Suboptimally Controlled Symptoms of Schizophrenia. Journal of Clinical Psychopharmacology: 7/8 2019-Volume 39—Issue 4—p 318-328.
Food and Drug Administration (U.S.). Discussion Document for Patient-Focused Drug Development Public Workshop on Guidance 3: SELECT, DEVELOP OR MODIFY FIT-FOR-PURPOSE CLINICAL OUTCOME ASSESSMENTS (including Appendices) (Published at Patient-Focused Drug Development Guidance: Methods to Identify What is Important to Patients and Select, Develop or Modify Fit-for-Purpose Clinical Outcome Assessments, Meeting Oct. 15-16, 2018) (“FDA 2018”).
Food and Drug Administration (U.S.). Patient-Focused Drug Development: Methods to Identify What Is Important to Patients Guidance for Industry, Food and Drug Administration Staff, and Other Stakeholders (October 2019) (“FDA 2019”).
Food and Drug Administration (U.S.). Patient-Focused Drug Development: Collecting Comprehensive and Representative Input; Guidance for Industry, Food and Drug Administration Staff, and Other Stakeholders (June 2020) (“FDA 2020”).
Guy W. ECDEU Assessment Manual for Psychopharmacology-Revised (DHEW Publication No. ADM 76-338). Rockville, M D, US Department of Health, Education, and Welfare, 1976:218-22.
Heffernan M L R, Herman L W, Brown S, Jones P G, Shao L, Hewitt M C, Campbell J E, Dedic N, Hopkins S C, Koblan K S, and Xie L. Ulotaront: A TAAR1 Agonist for the Treatment of Schizophrenia. ACS Med. Chem. Lett. 2022, 13, 92-98.
Keller A, McGarvey E L, Clayton, A H. Reliability and construct validity of the changes in sexual functioning questionnaire short-form (CSFQ-14), J Sex Marital Ther, 2006; 32(1): 43-52.
Koike H, Chaki S. Requirement of AMPA receptor stimulation for the sustained antidepressant activity of ketamine and LY341495 during the forced swim test in rats. Behav Brain Res 2014; 271:111-115.
Lam R W, Kennedy S H, Grigoriadis S, et al., Canadian Network for Mood and Anxiety Treatments (CANMAT). Canadian Network for Mood and Anxiety Treatments (CANMAT) clinical guidelines for the management of major depressive disorder in adults. III: pharmacotherapy. J Affect Disord 2009; 117 Suppl. 1: S26-43.
Lucki I, Dalvi A, Mayorga A J. Sensitivity to the effects of pharmacologically selective antidepressants in different strains of mice. Psychopharmacology. (Berl) 2001; 155:315-322.
Malhi G S, Adams D, Porter R, et al., Northern Sydney Central Coast Mental Health Drug & Alcohol, NSW Health Clinical Redesign Program, CADE Clinic, University of Sydney. Clinical practice recommendations for depression. Acta Psychiatr Scand Suppl 2009; 119 (439): 8-26.
Marcus R N, McQuade R D, Carson W H, Hennicken D, Fava M, Simon J S, Trivedi M H, Thase M E, Berman R M. The efficacy and safety of aripiprazole as adjunctive therapy in major depressive disorder: a second multicenter, randomized, double-blind, placebo-controlled study. J Clin Psychopharmacol. 2008 April; 28(2):156-65.
Montgomery S A, Asberg M. A new depression scale designed to be sensitive to change. Br J Psychiatry. 1979; 134:382-389.
Simpson G N, Angus J W S. A rating scale for extrapyramidal side effects. Acta Psychiatr Scand. 1970; 212(Suppl 44):S11-S19. US 2020/0179336 A1 (Jun. 11, 2020).
Williams J B. A structured interview guide for the Hamilton Depression Rating Scale. Arch Gen Psychiatry. 1988; 45:742-747.
Williams J B W. Structured interview guide for the Hamilton Anxiety Scale (SIGH-A). Biometrics Research Department, New York State Psychiatric Institute, New York, New York; Revision 15 Jan. 2008.
Willner P, Belzung C. Treatment-resistant depression: are animal models of depression fit for purpose? Psychopharmacology (Berl) 2015; 232(19):3473-3495.

Throughout this application, various publications are referenced. The disclosures of these publications in their entireties are hereby incorporated by reference into this application in order to more fully describe the state of the art to which this disclosure pertains. It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

1. A method of adjunctively treating MDD (major depressive disorder) in a human subject in need thereof who has exhibited an inadequate response to antidepressant therapy, comprising administering the antidepressant therapy and a therapeutically effective amount of ulotaront or a pharmaceutically acceptable salt thereof to the subject.

2. The method of claim 1, wherein inadequate response is defined, after at least 8 weeks of antidepressant therapy, as <50% reduction in depressive symptom severity, as assessed by the Massachusetts General Hospital Antidepressant Treatment Response Questionnaire (MGH-ATRQ).

3. The method of claim 1, wherein the antidepressant therapy comprises a SSRI (Selective Serotonin Reuptake Inhibitor) or an SNRI (Serotonin-norepinephrine Reuptake Inhibitor).

4. (canceled)

5. The method of claim 1, wherein:
a) the subject is suffering from a major depressive episode (MDE);
b) the subject has a history for the current MDE of an inadequate response to at least 1 and no more than 3 adequate antidepressant therapies (ADTs); and
c) the subject has a 17-item Hamilton Depression Rating Scale (HAM-D₁₇) total score≥18.

6. The method of claim 1, wherein inadequate response is defined, after at least 8 weeks of antidepressant therapy, as:
a) (i) a <50% reduction in the HAM-D17 (17-item Hamilton Depression Rating Scale) total score; or (ii) a HAM-D17 total score≥14;
b) a CGI-C (Clinical Global Impression—Change) Scale score of ≥3; and
c) a <50% reduction in MADRS.

7. The method of claim 1, wherein inadequate response is defined, after at least 8 weeks of antidepressant therapy, as:
a) a <50% reduction in the HAM-D17 (17-item Hamilton Depression Rating Scale) total score;
b) a HAM-D17 total score≥14;
c) a CGI-C (Clinical Global Impression—Change) Scale score of ≥3; and
d) a <50% reduction in MADRS total score.

8. The method of claim 1, wherein inadequate response is defined as <50% and >25% reduction in MADRS total score.

9. The method of claim 1, wherein the subject has anxious distress.

10. The method of claim 1, comprising administering the ulotaront or a pharmaceutically acceptable salt thereof and improving the subject's depression symptoms for a therapeutically effective period of time of 6 weeks or more, 8 weeks or more, 12 weeks or more, 26 weeks or more, or 52 weeks or more.

11. The method of claim 1, wherein the therapeutically effective amount comprises from 25 to 100 mg of ulotaront or a pharmaceutically acceptable salt thereof, based on the weight of the free form of ulotaront.

12. The method of claim 1, wherein the therapeutically effective amount comprises 50 or 75 mg of ulotaront or a pharmaceutically acceptable salt thereof, based on the weight of the free form of ulotaront.

13. The method of claim 1, wherein the subject has exhibited the inadequate response after at least eight weeks of the antidepressant therapy.

14. The method of claim 1, comprising administering the ulotaront or a pharmaceutically acceptable salt thereof for a period of at least six weeks.

15. The method of claim 1, wherein the method produces remission of the depression.

16. (canceled)

17. The method of claim 1, wherein the method improves the subject's MADRS total score; or wherein the method reduces the subject's MADRS total score greater than antidepressant therapy alone by ≥1, >2, >3, or ≥4 points; or wherein the method reduces the subject's CGI-S(Clinical Global Impression—Severity) score by ≥2, ≥3, or ≥4 points.

18-19. (canceled)

20. The method of claim 1, wherein the method produces a clinically significant improvement in the subject's MADRS (Montgomery Asberg Depression Rating Scale) total score and CGI-S(Clinical Global Impression—Severity) score.

21. The method of claim 1, wherein the method:
a) improves the subject's FAST (Functional Assessment Short Test) score; or
b) improves the subject's PGI-S(Patient Global Impression—Severity) score; or
c) improves the subject's SF-36 (36-Item Short-form Survey) score; or
d) improves the subject's HAM-A (Hamilton Anxiety Rating Scale) total score; or
e) improves the subject's HAM-D17 (17-item Hamilton Depression Rating Scale) total score; or
f) produces a CGI-C (Clinical Global Impression—Change) score of 1 or 2 (very much improved or much improved).

22-26. (canceled)

27. The method of claim 1, wherein the method decreases the subject's MADRS total score by ≥50% and/or reduces the subject's MADRS total score to ≤10.

28-29. (canceled)

30. The method of claim 1, wherein the method results in no clinically significant worsening of the subject's:
a) abnormal involuntary movements as measured by the AIMS (Abnormal Involuntary Movement Scale);
b) akathisia as measured by the BARS (Barnes Akathisia Rating Scale);
c) sexual functioning as measured by the CSFQ-14 (Changes in Sexual Functioning Questionnaire, Short Form);
d) suicidality as measured by the C-SSRS (Columbia-Suicide Severity Rating Scale);
e) Parkinsonism as measured by the SAS (Simpson Angus Scale);
f) sleep quality as measured by the PSQI (Pittsburgh Sleep Quality Index); or
g) any combination of two or more of (a)-(f)
wherein the method results in no clinically significant sedation, symptoms of metabolic syndrome selected from weight gain, diabetes, and hyperlipidemia, or movement disorders.

31. (canceled)

32. The method of claim 1, further comprising monitoring for signs of:
i) CNS (central nervous system) stimulation comprising agitation, restlessness, and/or insomnia;
ii) depression comprising lethargy, somnolence, and/or shallow breathing; or
ii) impaired muscle coordination, effects on heart rate, and/or change in pupil size.

33. (canceled)
